(19) United States
(12) Patent Application Publication
(10) Pub. No.: US 2014/0051099 A1
(43) Pub. Date: Feb. 20, 2014

(54) DIAGNOSIS AND TREATMENT OF ANGIOEDEMA

(71) Applicants: Christian Drouet, Meylan (FR); Samuel Luyasu, Saint-Remy (BE); Arije Ghannam, Grenoble (FR)

(72) Inventors: Christian Drouet, Meylan (FR); Samuel Luyasu, Saint-Remy (BE); Arije Ghannam, Grenoble (FR)

(73) Assignees: CENTRE HOSPITALIER UNIVERSITAIRE DE GRENOBLE, Grenoble (FR); UNIVERSITE JOSEPH FOURIER (GRENOBLE 1), Grenoble (FR)

(21) Appl. No.: 14/111,952

(22) PCT Filed: Apr. 12, 2012
(86) PCT No.: PCT/FR12/50804

§ 371 Date: Oct. 31, 2013

(30) Foreign Application Priority Data
Apr. 14, 2011 (FR) 11/53239

(51) Int. Cl. G01N 33/68

(52) U.S. Cl. CPC G01N 33/6893
USPC 435/7.93

(57) ABSTRACT

The present patent application relates to a method for diagnosing angioedema with a gain-of-function mutation of kinin formation, and for determining the amount of BK or des-Arg⁹ -BK antagonist required for halting the effects of angioedema, from a plasma sample of a patient. The invention also relates to a kit for said determination.

The present patent application concerns a method for diagnosing angioedema with gain-of-function kinin-forming capacity and bradykinin production (BK) during an attack, to determine the required quantity of BK antagonist for halting the effects of angioedema from a patient's plasma sample. The invention also concerns a kit for this determination.

Angioedema (AE) is a pathological situation expressed by capillary leakage, with swelling of sub-cutaneous or sub-mucosal tissues. This capillary leakage is generated by the production of BK via the cleavage of its precursor, high molecular weight kininogen (HK), by plasma kallikrein. BK exerts its effects by stimulating the kinin B2 receptor expressed on endothelial cells. Vascular permeability in a patient undergoing an attack leads to abdominal cramps sometimes accompanied by vomiting and diarrhoea. The attack also causes respiratory problems which may go as far as asphyxia if it is located in the larynx region. In some severe cases angioedema may lead to the patient's death.

Patients undergoing an attack must be given emergency treatment. Diagnosis is difficult since it has symptoms common with allergy, oedema and anaphylactic shock. One of the emergency treatments for these patients is icatibant, an antagonist of the B2 receptor of BK, via sub-cutaneous route in the abdominal region. The injection is intended to halt the effects of the attack. Icatibant is packaged in the form of a 30 mg solution for injection (10 mg/ml). This packaging represents an excess quantity. This is chiefly due to the fact that no method for determining the amount of icatibant needed to halt the effects of an attack is currently available.

Despite this lack of knowledge, to inject an excess amount of icatibant presently ensures the efficacy of emergency treatment for a patient undergoing an acute attack. Nonetheless this also entails a certain number of disadvantages. One of these is the onset in the patient of adverse side effects and in particular reactions at the injection sites (redness, burning sensation, pain) in almost all patients. Another disadvantage is recurrence of an attack in some patients. Icatibant also has a very high retail price and there is no doubt that a more adapted dosage would reduce the cost of treatment and hence the health expenditure incurred.

It would therefore be particularly advantageous to have available a method which, on a case-by-case basis from a blood sample of the patient and in relation to the severity of the attack, which allows determination of the quantity of icatibant needed to halt the effects of this attack. The present invention comes within this context.

Lack of diagnosis based on the metabolism of kinin formation also leads the present invention to developing in this direction.

The present invention therefore allows the diagnosis, follow-up, prognosis and treatment of gain-of-function angioedema.

### DESCRIPTION OF THE INVENTION

The present invention concerns a method for determining the required quantity of BK antagonist to halt the effects of angioedema in a patient, comprising:

a) activating the release of BK by contacting a plasma sample of the patient with a determined quantity of electronegative agent;

b) assay of the quantity of BK released at step a), which corresponds to the BK reserve in the patient's plasma sample;

c) comparing the value obtained at step b) with a control value which corresponds to the BK reserve available in a plasma sample of a healthy control of same category, to determine the amount of BK produced in the patient's plasma sample;

d) determining the total quantity of BK produced in the patient; and

e) determining the necessary quantity of said antagonist from the determination performed at step d) this quantity being equimolar with the quantity of BK produced.

According to one aspect of the invention, the assay step b) is a competitive immunoenzymatic assay.

According to another aspect of the invention, for the assay at step b) use is made of anti-BK antibodies, DIG tracers coupled to BK, anti-DIG antibodies coupled to an enzyme and substrates of the enzyme.

According to yet another aspect of the invention, step b) itself comprises the following steps:
anti-BK antibodies diluted in binding buffer are immobilised on a suitable support comprising several cavities;
at regular time intervals an aliquot portion of the activated sample is taken and a cavity of the support is incubated with said aliquot portion of the sample and a determined quantity of DIG tracer coupled to BK for a determined period of time;
after washing, the support is incubated with anti-DIG antibodies coupled to an enzyme;
after washing, the support is incubated with a determined quantity of the enzyme substrate;
the enzymatic activity is measured by direct colorimetric measurement.

The enzyme coupled to the anti-DIG antibodies may be alkaline phosphatase or peroxidase for example.

According to one aspect of the invention, the BK antagonist is icatibant (HOE 140).

The invention also concerns a kit to determine the amount of BK antagonist needed to halt the effects of angioedema in a patent, using a sample of whole blood from the said patient, comprising:
a first reagent consisting of anti-BK antibodies;
a second reagent consisting of a DIG tracer coupled to bradykinin;
a third reagent consisting of anti-DIG antibodies coupled to an enzyme;
a fourth reagent consisting of a substrate of the enzyme.

The invention also concerns a method for diagnosing gain-of-function angioedema in a patient, comprising:

a) activating the release of BK by contacting a plasma sample of the patient with a determined quantity of electronegative agent;

b) assay of the quantity of BK released at step a) which corresponds to the BK reserve in the patient's plasma sample;

c) comparing the value obtained at step b) with a control value which corresponds to the BK reserve available in a plasma sample of a healthy control of same category, to determine the amount of BK produced in the patient's plasma sample; and

d) determining the total quantity of BK produced in the patient.

### DESCRIPTION OF THE FIGURES

FIG. 1: Formation of endothelial bradykinin (kinin formation).

FIG. 2: Kinin-forming capacity in patients suffering from angioedema. The plasma is subjected to in vitro activation by a charged surface (glass beads) and the concentrations of BK (A) and desArg⁹-BK (B) are monitored over time (over 60 minutes). AE+: population suffering from angioedema subsequent to therapy with angiotensin-1 converting enzyme inhibitors; AE-: normal population without any pathological sign and subjected to the same therapy; reference population: normal reference population.

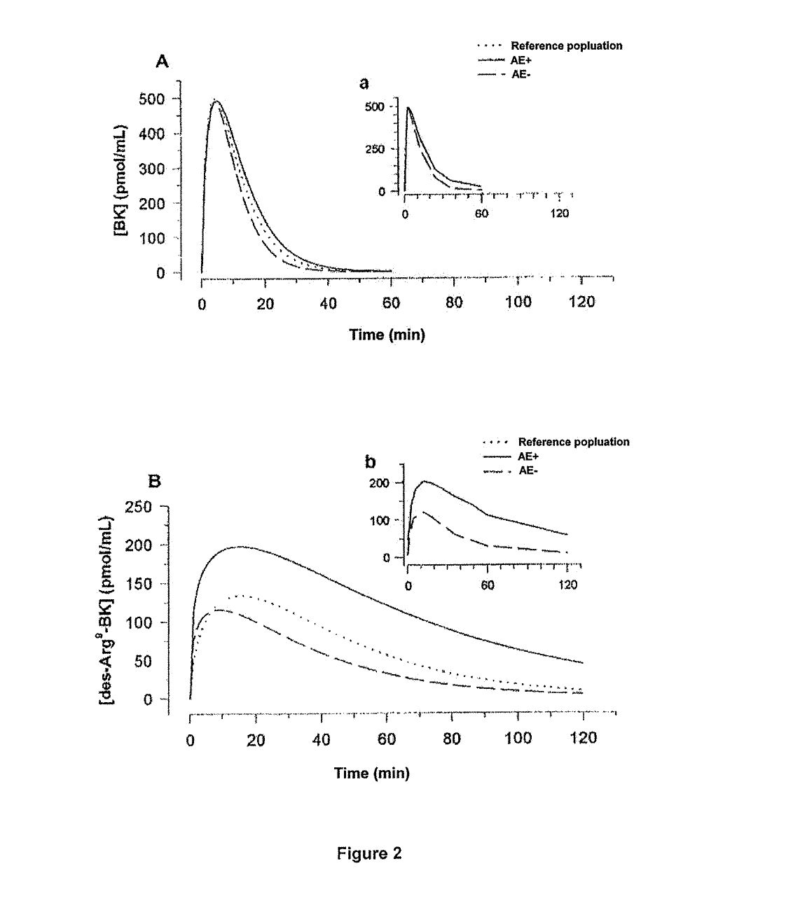

FIG. 3: Biological events (spontaneous kininogenase and activatability potential) before the attack, during the attack and 21 days after an attack in a patient.

FIG. 4: Trend in the capacity of the HK kininogen to produce BK and desArg⁹-BK in two situations: before and after the attack of gain-of-function angioedema (FIGS. 4A, 4B).

Method for Determining the Quantity of Bradykinin Antagonist Needed to Halt the Effects of Angioedema.

The present invention therefore concerns a method for determining the quantity of BK antagonist required to halt the effects of angioedema in a patient. This method comprises five essential steps. The method of the invention uses a patient's citrated plasma sample. For example the volume of the patient's plasma sample is 500 μl.

At a first step, the release of BK is activated by contacting the patient's plasma sample with a determined quantity of electronegative agents. This results in activating the proteases of the contact phase, with cleavage of the HK kininogen present in the plasma. This cleavage concerns two peptide bonds (Sequence ID P01042—before the Arginine residue at position 380 and after the Arginine residue at position 388) to yield BK.

By <<patient>> is meant a human individual in a situation of undergoing an angioedema attack, or who is suspected of being in the course of preparing an angioedema attack.

By <<electronegative agent>> is meant an agent able to activate the plasma contact phase, the BK production system. It may be a solid product or a soluble product for example. Mention can be made of the use of dextran sulfate, glass beads or aluminium silicate (kaolin, Al₂Si₂O₅(OH₄).

By <<determined quantity of electronegative agents>> is meant the quantity of electronegative agents needed to activate the contact phase in the volume of plasma under consideration in the experiment; this quantity is in excess relative to the quantity of pro-enzymes of the contact phase and is determined by observing the dose-response curve (Kluft, 1978; Müller, 2009). If for example the choice of electronegative agent concerns dextran sulfate, the required concentration of electronegative agent is 25 μg/ml.

At a second step of the method of the invention, the assay is performed of the quantity of BK released at step a), which corresponds to the BK reserve in the patient's plasma sample. By <<BK reserve>> is meant the amount of BK available in the plasma sample i.e. which can be released by activation of the contact phase. Comparison of this value with a control value (step c) of the method of the present invention allows the evaluation of the amount of BK produced by the patient during the pathological event, and hence the determination of the quantity of antagonist which must be given to the patient to halt the effects of the attack.

This quantity of BK may in particular be expressed in unit weight per unit of plasma volume (for example in ng/ml) or in the number of moles per unit plasma volume (for example in nmol/l).

According to one aspect of the invention, the BK assay is performed by competitive immmunoenzymatic assay.

By <<competitive immunoenzymatic assay>> is meant a quantitative assay including measurement of a calibrated standard and the performing of a competitive reaction against the standard using a tracer.

By <<tracer>> is meant an entity mimicking the antigen standard and to which a molecule is attached allowing the tracing thereof. Tracers are produced for example by coupling the antigen to DIG (digoxigenin) at the amino-terminal part.

According to one aspect of the invention, the assay of the quantity of BK comprises one or more steps. When this assay is performed in several steps, the plasma sample and the tracers are placed in contact with an antibody (capture antibody) specifically recognising BK. The captured antigen (including the BK to be assayed and the tracer) is placed in contact with an antibody (detection antibody). If the assay is performed in a single step, the tested antigen (including the BK to be assayed and the tracer) is contacted with the detection antibody simultaneously in the well containing the capture antibody.

Specific antibodies can be obtained using the techniques described for example in Czernik A J, et al (1991), Production of phosphorylation state-specific antibodies. Methods Enzymol 201: 264-283. For this purpose, animals e.g. rabbits can be immunised with the carboxy-terminal portion of BK which must be sufficient to form an epitope. Preferably antibodies are chosen which have high affinity and good specificity for the antigen.

If necessary, the assay can be performed using a second antibody which recognises the first antibody, to improve specificity.

By <<antibody>> is meant an antibody comprising all the immunoglobulins or a fragment of these immunoglobulins containing or formed by the recognition site for the target antigen, for example a fragment formed by a heavy chain or by the association of heavy and light chains or a fragment thereof e.g. a (Fab)′2 or Fab fragment.

Detection of the formation of the immunological complex is performed using a substrate of the antibody marker e.g. a stained substrate or fluorescent substrate and comprises a step to measure optical density. A marker such as peroxidase or alkaline phosphatase can be used.

Measurement of optical density is conducted for example at 450 nm, in particular if the antibody marker is a peroxidase.

Antibodies specific to BK or desArg⁹-BK are commercially available or can be prepared using methods cited in the present application.

For example, the following antibodies are available from the following companies:

(1) antibodies-online GmBH, SchlossRaheStr. 15, D-52072 Aachen, Germany, Fax: +33 8111 49 891, Tel: +33 8111 49 890.

(2) Abcam, 24 rue Louis Blanc, 75010 Paris.

(3) AbDSerotecMorphoSys UK Ltd., Endeavour House, Langford Business Park, LangfordLane, Kidlington, Oxford GB-OX5 1GE, UK.

(4) Bachemdistribution Services GmbH, HegenheimerStrasse 5, D-79576 Weil am Rhein, Germany.

To carry out the assay according to the invention, the blood sample taken from a patient is treated to prevent possible coagulation thereof. In particular, a usual anticoagulant agent is added thereto to perform in vitro assays on the blood, for example heparin or sodium citrate, such as trisodiumcitrate for example at concentrations of 0.109 M or 0.129 M following the proportions of 9 volumes of blood per 1 volume of anticoagulant.

It is possible for example for the BK assay to use anti-BK antibodies, DIG tracers coupled to BK, anti-DIG antibodies coupled to an enzyme and substrates of the enzyme.

According to one embodiment of the invention the BK assay comprises the following steps:
anti-BK antibodies diluted in binding buffer are immobilised on a suitable support comprising several cavities; by suitable support is notably meant a solid plate e.g. a 96-well plate;
at regular time intervals an aliquot portion of the activated sample is taken and a cavity of the support is incubated with said aliquot portion of the sample and a determined quantity of DIG tracer coupled to BK for a determined period of time; for example the following quantities of tracers are used: 10-20 pg of BK-DIG (0.1-0.6 pg of desArg⁹-BK-DIG for the assay of desArg⁹-BK);
after washing, the support is incubated with anti-DIG antibodies coupled to an enzyme; for example the enzyme coupled to the anti-DIG antibodies is alkaline phosphatase or HRP peroxidase;
after washing, the support is incubated with a determined quantity of substrate of the enzyme;
enzymatic activity is measured by direct colorimetric measurement.

For example the assay is performed as follows: first the anti-BK immunoglobulins G are deposited in the wells of a 96-well plate. Next, the sample to be assayed or the standard is left to compete with a known quantity of BK coupled to the tracer (DIG). Anti-DIG antibodies coupled to HRP are then deposited in each well. Finally the presence of immune complex is detected by measuring the activity of peroxidase. More specifically, a solution containing o-phenylenediamine is incubated in each well. Absorption of the oxidised substrate is read at 450 nm.

At a third step of the method of the invention, a comparison, more specifically a subtraction, is made between the quantity of released BK (value obtained at step b) of the method of the invention) and the control value which corresponds to the BK reserve available in a plasma sample of a healthy control of same category, the volume of the plasma sample of the control being identical to that of the patient's sample. For example, if the assay at step b) was conducted using a plasma volume of 500 μL, the comparison must be made with a control value obtained from an identical volume i.e. 500 μL.

By <<healthy control of same category>> is meant a control not in a situation of an angioedema attack. This control must be of same category i.e. of same gender and of approximately identical weight (in kg) and height (in cm), variations to within 10% being acceptable however. This control may be a different individual or it may be the same person if the assay was performed at a time when the patient was not undergoing or in the preparation of an attack.

Therefore, at this step the BK reserve in a volume of patient's plasma sample is compared with the BK reserve in an identical volume of plasma of a healthy control of same category. In other words, the patient's BK reserve is compared with a control value. More specifically, this allows the evaluation of the quantity of BK that is mobilised by the angioedema attack process, a pathological event, in a patient as compared with a healthy control.

Therefore the quantity of BK produced in the patient's plasma sample is determined from the value obtained at the assay step of the patient's sample (patient reserve) and from the control value in an identical plasma volume of a healthy control (healthy control reserve). In other words, the subtraction is performed between the quantity of patient BK reserve and the quantity of control BK reserve, so as to evaluate the quantity of BK that the patient has produced during the pathological event. This quantity of BK can notably be expressed in unit weight per unit volume of plasma (for example in ng/ml) or in number of moles per unit volume of plasma (for example in nmol/l).

At a fourth step, the total quantity of BK produced in the patient is determined. It is a step which consists of evaluating the quantity of BK produced in the total plasma volume of the patient (taking into account the patient's weight, blood volume and plasma volume) from the determination performed at the preceding step i.e. from the quantity of BK ina known volume of plasma.

At this step, the value obtained at step three which corresponds to a quantity of BK in a determined volume of plasma sample e.g. 500 μL, is related to the total plasma volume of the patient e.g. 2.25 L using the rule of three.

This quantity can be expressed in particular in weight (for example in mg) or number of moles (for example in nmol). At a fifth step, the required quantity of antagonist is determined from the determination made at step four, this quantity being equimolar with the consumed quantity of BK.

In other words, the number of moles of antagonist required to halt the effects of angioedema in a patient is equal to the number of moles of BK determined at step four of the method of the invention. This number of moles is then converted to weight of antagonist which must be given to the patient taking into account the molecular weight of the antagonist. For example the molecular weight of the antagonist icatibant is 1305 g/mol.

Method for Determining the Quantity of Antagonist Needed to Halt the Effects of Angioedema

The present invention also concerns a method for determining the quantity of antagonist of desArg⁹-BK required to halt the effects of angioedema in a patient, comprising the following steps:

a) the release of desArg⁹-BK is activated by contacting a sample of the patient's plasma with a determined quantity of electronegative agent;

b) an assay is performed of the quantity of desArg⁹-BK released at step a), which corresponds to the desArg⁹-BK reserve in the patient's plasma sample;

c) the value obtained at step b) is compared with a control value which corresponds to the desArg⁹-BK reserve available in a plasma sample of a healthy control of same category, so as to determine the quantity of desArg⁹-BK produced in the patient's plasma sample;

d) the total quantity of desArg⁹-BK produced in the patient is determined; and

e) the required quantity of said antagonist is determined from the determination performed at step d), this quantity being equimolar with the quantity of desArg⁹-BK produced.

According to this aspect of the invention, the assay at step b) is a competitive immunoenzymatic assay.

According to another aspect of the invention, for the assay at step b) use is made of anti-desArg⁹-BK antibodies, DIG tracers coupled to desArg⁹-BK, anti-DIG antibodies coupled to an enzyme and substrates of the enzyme.

According to yet another aspect of the invention, step b) itself comprises the following steps:
anti-desArg⁹-BK antibodies diluted in binding buffer are immobilised on a suitable support comprising several cavities;
at regular time intervals an aliquot portion of the activated sample is taken and a cavity of the support is incubated with said aliquot portion of the sample and a determined quantity of a DIG tracer coupled to desArg⁹-BK, for a determined period of time;
after washing, the support is incubated with anti-DIG antibodies coupled to an enzyme;
after washing, the support is incubated with a determined quantity of substrate of the enzyme;
enzymatic activity is measured by direct colorimetric measurement.

The enzyme coupled to the anti-DIG antibodies may be alkaline phosphatase or HRP peroxidase for example.

According to one aspect of the invention, the antagonist of desArg⁹-BK is R954 i.e. the peptide Ac-Om-[Oic², αMePhe⁵, D-βNal⁷, IIe⁸]desArg⁹-BK.

### Method for Diagnosing Gain-of-Function Angioedema

The present invention also concerns a method for diagnosing gain-of-function angioedema in a patient. This method comprises the following steps:

a) the release of BK is activated by contacting a patient's plasma sample with a determined quantity of electronegative agent;

b) the quantity of BK released at step a) is assayed, which corresponds to the BK reserve in the patient's plasma sample; and

c) the value obtained at step b) is compared with a control value which corresponds to the BK reserve available in a plasma sample of a healthy control of same category, to determine the quantity of BK produced in the patient's plasma sample; and

d) optionally, the total quantity of BK produced in the patient is determined.

By <<patient>> is meant any individual in whom qualitative approximation of the capacity to produce kinins from the HK kininogen is necessary for diagnosis of susceptibility.

### Kit

The present invention also concerns a kit to implement one of the determination methods according to the present invention, comprising:
a first reagent consisting of anti-BK or anti-desArg⁹-BK antibodies;
a second reagent consisting of a DIG tracer coupled to BK or coupled to desArg⁹-BK;
a third reagent consisting of anti-DIG antibodies coupled to an enzyme;
a fourth reagent consisting of a substrate of the enzyme.

### EXAMPLES

### Example 1

### Spontaneous Kininogenase Activity and Activatability of Proenzymes

### Materials and Methods

a) Sampling

Citrate plasma: 2×4.5 mL of blood were taken by vein puncture and collected in BD Vacutainer® tubes, light-blue caps, containing 0.1 mol/L Na citrate (type 366415). The plasma was collected after centrifuging (20° C., 15 min, 2500×g).
b) Plasma kininogenase Activity

Principle: the activity of the enzymes cleaving HK kininogen (kininogenases) was estimated against the peptide Pro-Phe-Arg-para-nitroanilide, representing the C-terminus of BK (residues 285-289 of HK; identified peptide sequence P01042). The corresponding proenzymes can be activated by electronegative agents (e.g. dextran sulfate). It is evaluated using an amidolytic method using the H-D-Pro-Phe-Arg-pNA substrate whose cleavage is detectable by colour change (release of the chromophore-para-nitroanilinep-NA group) allowing photometric measurement at 405 nm (c 405 nm=8800 M⁻¹ .cm⁻¹).

Two activities were measured on the plasma samples:

1—Spontaneous activity of the plasma

2—Degree of proenzymeactivatability by dextran sulfate.

TABLE 1
Reference values of enzymatic activities of kinin formation

| | Plasma | | |
|---|---|---|---|
| | spontaneous | D.S. | |
| | kininogenase | activatability | |
| | activity | potential | |
| | (nmol/ml/min) | (nmol/ml/min) | Ratio (%) |
| Male subject | (2.3-5.6) | (2225-4273) | (0.07-0.17) |
| (18-58 years) | median 3.6 | 3288 | 0.11 |
| Female subject | (2.4-10.7) | (2422-4560) | (0.07-0.29) |
| (18-57 years) | median 4.3 | 3523 | 0.13 |

### Example 2

### Kinin-Forminq Capacity in Patients Suffering from Angioedema

Assay of BK and desArq⁹-BK kinins

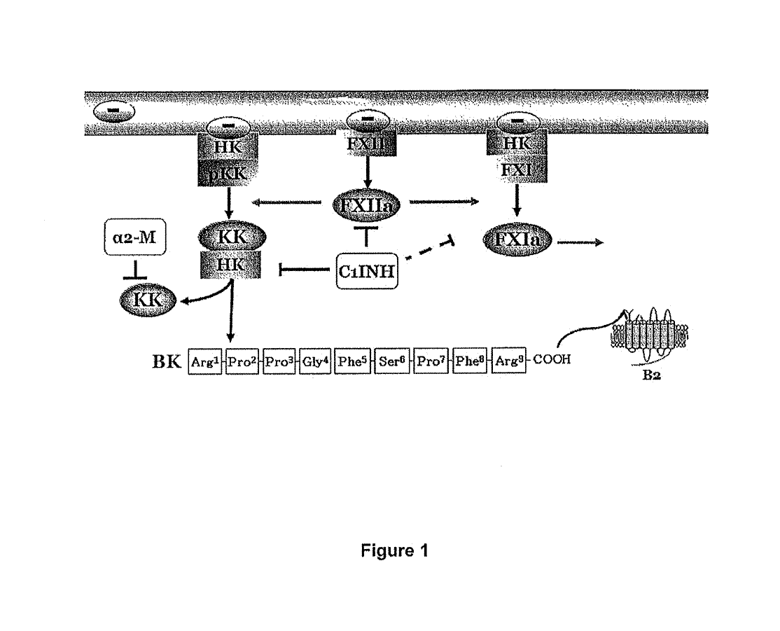

The kinin-forming capacity of the patients' plasmas was examined in the context of plasma samples from patients suffering from angioedema by measuring the kinetics of plasma BK production (FIG. 1A). This measurement can by supplemented by measurement of desArg⁹-BK which gives information on the catabolism of BK (FIG. 1 B).

### Material and Methods

The volume of plasma (500 μL) was activated in vitro by dextran sulfate (SIGMA, 25 pg/ml final concentration), an activator high in negative charges. At regular intervals (0 min, 1 min, 2 min, 4 min, 6 min, 8 min, 16 min, 32 min, 60 min) 50 μL were taken, precipitated with 10 volumes of ethanol and centrifuged (4° C., 30 min, 3500×g). After evaporation of the supernatants (Speed Vac) and dissolving of the content of the tubes in 50 pl of distilled water, the concentrations of BK (A) and desArg⁹-BK (B) were measured by ELISA competitive assay with the peptide tracer labelled with DIG(ROCHE) and using specific antibodies. Absorbance was measured by spectrophotometry, and the assays identified in relation to a standard range.

These data qualify angioedema and give access to strategic parameters on the ligands of the vascular permeability receptors:
the quantity (mol) of kinins (BK and desArg⁹-BK) produced from HK kininogen by assay of the BK reserve and comparison with a control value, more specifically by calculating the area under curve AUC (evaluation of the extent of cleavage of the HK kininogen);
the quantity of antagonist drug of the B2 receptor (icatibant) required to displace the BK produced;
the BK half-formation time by the patient's plasma (evaluation of kinin formation);
the half-life of each of the kinins in the plasma medium (evaluation of catabolism).

### Example 3

### Patient Undergoing an Attack

1. Clinical Situation

A female patient aged 24 years was admitted to the emergency department for angioedema. She presented with abdominal cramps, respiratory problems, eyelid angioedema.

The patient did not carry the mis-sense 983A/G mutation of exon 9 of the F2 gene, was not undergoing medication, normal C1 lnh function.

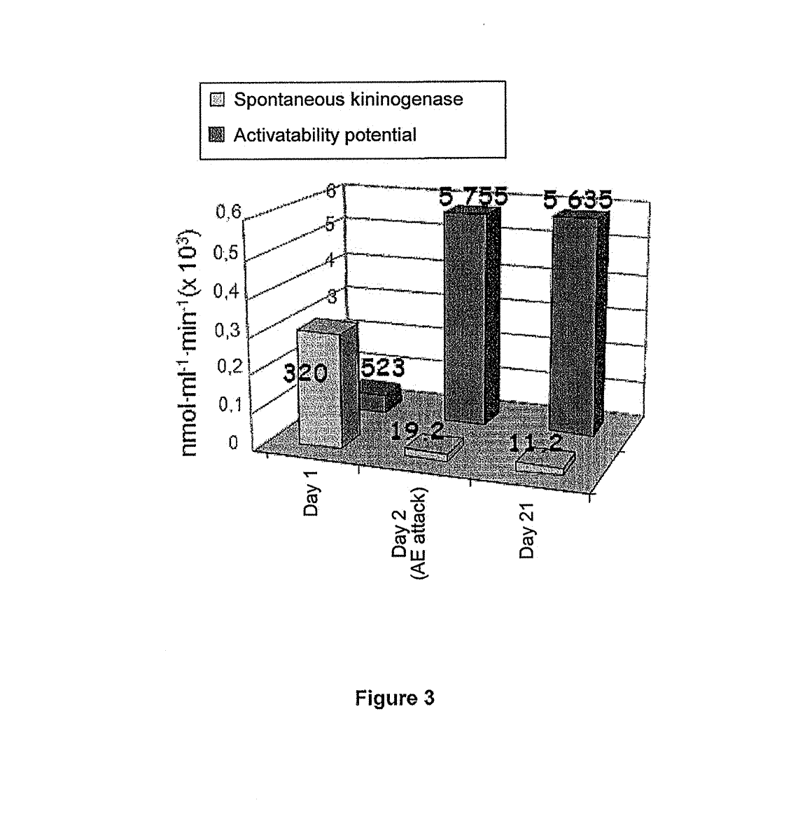

2. Biology
2.1 Strong kinin formation with high spontaneous activity, normal kinin catabolism (Table 2)
2.2 Kinetics of biological events before attack, during attack and after resolution (FIG. 3).

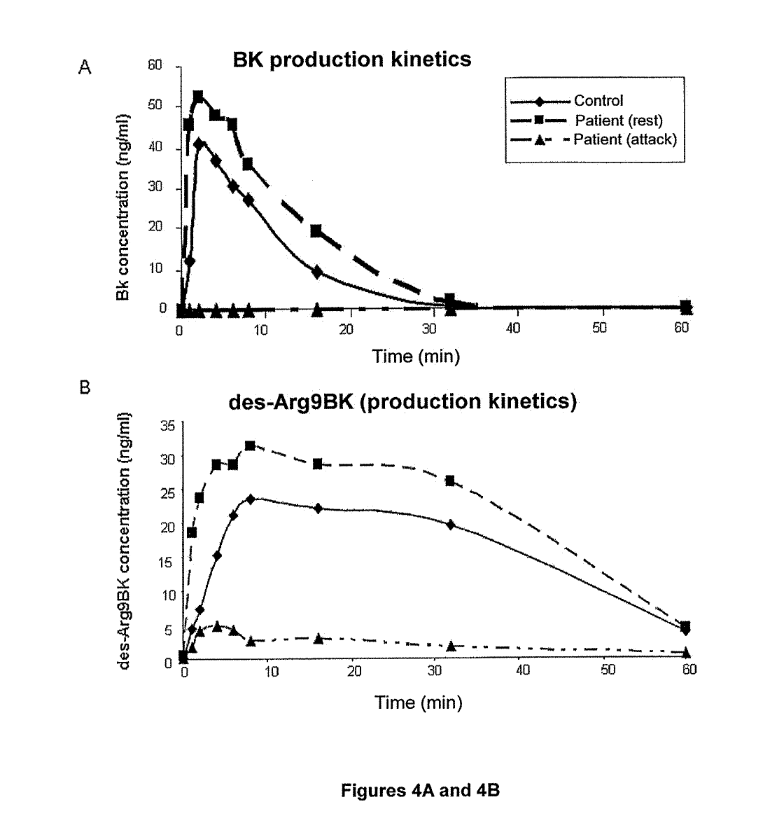

Observation: The proenzymes were rapidly reconstituted via hepatic synthesis after activation, with a half-reconstitution time of less than 6 hours.
2.3 Trend in the capacity of the HK kininogen to produce BK and desArg⁹-BK in both situations: before and after the gain-of-function angioedema attack (FIGS. 4A 4B)

Observation: the HK kininogen undergoes extensive cleavage by endothelial proteases. This cleavage during an angioedema attack translates as loss of production of BK and desArg⁹-BK. This indicates functional loss of the HK kininogen. The function and integrity of the HK kininogen were slowly restored with a half-reconstitution time of more than 48 hours.

### 3. Results

### 3.1 Spontaneous Kinin-Formation: High Spontaneous Activity

TABLE 2
Enzymatic activities of kinin formation
during clinical events of angioedema.

| | Plasma | | |
|---|---|---|---|
| | spontaneous | D.S. | |
| | kininogenase | activatability | |
| | activity | potential | |
| | (nmol/ml/min) | (nmol/ml/min) | Ratio (%) |
| Sample N° 1 | 320 | 523 | 36.98 |
| Taken at | | | |
| consultation | | | |
| Sample N° 2 | 19.2 | 5 755 | 0.33 |
| Taken during | | | |
| attack, 33 h after | | | |
| 1^{st} sample | | | |
| Sample N° 3 | 11.2 | 5 635 | 0.20 |
| Control taken after | | | |
| 21 days | | | |

The activities of the enzymes of BK and desArg⁹-BK kinin catabolism were normal (data not given).

To evaluate the possible contribution of mastocytes, the tryptase level was investigated in plasma samples 1 and 2:
3.65 pg/I (reference <13.5 μg/l)
4.30 pg/I (reference <13.5 μg/l) and in a sample 3 independent of the attack: 3.1 μg/l (reference <13.5 μg/l).

There was no change in tryptase level over time, which excludes any mastocyte participation.

2. Kinetics of Biological Events before the Attack, During the Attack and 21 Days after the Attack.

These concerned the first observation of enzyme and kinin development during the angioedema attack and after this attack, in a non-C1 lnh-deficient subject not carrying the mis-sense c.983A/G mutation (p. Thr328Lys) on the F12 gene. Originality: first observation of kinin formation during the hours preceding symptomatology and during symptomatology.

### 3. Interpretation of Experimental Data

Kinin formation is a pre-condition of an attack, about 24-36 hours before oedema. Spontaneous kininogenase activity with the production of BK is an early diagnosis factor anticipating the clinical event.
The rapid reconstitution of the proenzymes is coherent with strong hepatic production, rapidly mobilising prekallikrein and the Hageman factor proenzyme at endothelial level.
The decrease in proenzymeactivatability provides solid ex vivo evidence of the rapid endothelial activation of the contact phase proenzymes.
The normal tryptase level in the samples excludes any mastocyte component and associated proteases in this phenomenon.

### Explanation of the Biological Phenomenon:

The strong activation of the proenzymes translates as proteolysis of the HK kininogen.
The proteolytic activity with respect to the HK kininogen is transient; its degradation by proteolytic cleavage is very extensive.
Clinical-biological phenomenon: Consequently the diffusion of bradykinin and desArg⁹-BK causes activation of the B2 and B1 receptors and triggers vascular permeability.

### Medical Interpretation

The symptomatology of angioedema follows after the enzymatic activity capable of producing kinins; a period of 24-36 hours is the time needed for diffusion of water towards the tissues.
The present observation provides pedagogical documentation of clinical angioedema in a situation of kininogenase gain-of-function.

### CONCLUSION

These biological tests can be applied to examining a situation of gain-of-function angioedema.
They must be chosen to provide the necessary arguments in favour of determining etio-pathogenic diagnosis, needed to take the decision for applying targeted therapy.

### Example 4

### Patients in Attack-Diagnosis Situation and Determination of the Quantity of BK Antagonist Required to Halt the Effects of Angioedema

a) Female subject aged 24 years not carrying the mis-sense 983A/G mutation of exon 9 of the F12 gene, not undergoing any medication, normal C1Inh function.

Initially an assay is performed of the quantity of BK released (or produced) in a citrated plasma sample of the patient by in vitro activation of the contact phase.

The value obtained corresponds to the BK reserve or amount of available BK in the patient's whole blood sample (volume: 500 μL). This assay is conducted such as described in Example 2.

FIG. 4A shows that the residual production of BKBK is very low in a patient in a pathological situation (Sample 1, patient undergoing an attack), compared with the control.

FIG. 4A also shows that when the pathological situation is over (Sample 3, patient at rest) the patient has a BK production capacity comparable to that of the control.

At a second step the value obtained at step a) is compared with a control value corresponding to the assay of the BK reserve in a whole blood sample of a healthy control of same category (FIG. 4A, control). This control value corresponds to the area under curve AUC of BK (FIG. 4A).

Curves 4A of the patient and of the control are smoothed using the B-splines method. The areas under curve AUCs represent the quantity of BK released by activation of the contact phase; they are calculated with or without smoothing using the trapeze method.

| | After smoothing | Without smoothing |
|---|---|---|
| AUC 3A3 (patient in | 512.0 ng/ml | 751.75 ng/ml |
| situation at rest, | | |
| Sample 3) | | |
| AUC 3A1 (patient in | 2.74 ng/ml | 3.9 ng/ml |
| pathological situation), | | |
| Sample 1) | | |
| Control | 564.68 ng/ml | 465.18 ng/ml |

At a third step, the calculation of the differences between the situation at rest (Sample 3) and the pathological situation (Sample 1) leads to knowledge of BK production (ng/ml) produced by activation of the plasma sample. This makes it possible to calculate the quantity of BK (molecular weight 1060) produced in the patient's sample volume during pathological development.

| | |
|---|---|
| After smoothing | Without smoothing |

| | | | | |
|---|---|---|---|---|
| AUC 3A3 − AUC 3A1 | 509.2 | ng/ml | 747.8 | ng/ml |
| (patient) | 480.4 | nmol/l | 705 | nmol/l |

When related to total plasma volume, the difference AUC 3A3-AUC 3A1 therefore corresponds to the quantity of BK produced during the active phase of symptomatology: Patient weight 54 kg; Blood volume 4.1 I; Plasma volume 2.25 I.

| | |
|---|---|
| After smoothing | Without smoothing |

| | | | | |
|---|---|---|---|---|
| AUC 3A3 − AUC 3A1 | 1.15 | mg | 1.68 | mg |
| (patient) | 1 081 | nmol | 1 586.2 | nmol |

At this stage, the data are sufficient to state that the patient has gain-of-function angioedema.

Finally a determination is made of the required quantity of said antagonist. The quantity of medication to be applied must at least be 1081 nmoli.e. about 1.4 mg of icatibant antagonist (molecular weight 1305). Icatibant is packaged in the form of a 3 ml injectable solution of 10 mg/ml. This means that at the present time patients undergoing an attack and arriving at emergency departments are given a quantity of antagonist of 30 mg. The method of the present invention indicates that a much lower quantity is sufficient to halt the effects of angioedema. This also has the advantage of reducing the adverse effects associated with administering a quantity of icatibant that is too high.

b) Female subject aged 61 years, not carrying a mutation on the SERPING1 and F2 genes, developing angioedema pathology under an oestrogen agonist initiator (raloxifene); strong spontaneous kininogenase activity at the time of attack.

c) Female subject aged 33 years, not carrying a mutation on the SERPINGI and F12 genes, developing angioedema pathology under the oestrogenic initiator (ethinyl-oestradiol); strong spontaneous kininogenase activity at the time of attack.

The plasma samples of the subjects b) and c) were collected during and after the active period of the disease.

These were subjected to the method according to the invention for determining the quantity of BK antagonist required for halting the angioedema attack:
activation by dextran sulfate (25 μg/ml) as electronegative agent;
assay of the quantities of BK released over time during the 60 minutes following after activation (sample taken during the attack and sample taken after the attack which corresponds to a control value) and comparison of obtained values;
determination of the total quantity of BK produced;
determination of the quantity of antagonist needed to halt the angioedema attack.

| | Patient b) | | Patient c) | |
|---|---|---|---|---|
| Quantity of BK produced in patient's | 448.5 | ng/ml | 437 | |
| plasma sample | | | | |
| Total quantity of BK produced in the | 1.17 | mg | 1.08 | mg |
| patient | 1.10 | nmol | 1.01 | nmol |
| Quantity of antagonist needed to displace | 1.43 | mg | 1.32 | mg |
| produced BK, halt angioedema attack | | | | |

The quantity of medication to be given must be at least 1.43 mg of icatibant antagonist (molecular weight 1305) for patient b) and 1.32 mg for patient c).

### Example 5

### Patient Undergoing an Attack—Determination of the Quantity of desArg⁹-BK Antagonist Needed to Halt the Effects of Angioedema

The assay is performed of the quantity of desArg9-BK produced in a citrate plasma sample of the patient via in vitro activation of the contact phase.

FIG. 4B shows that the residual production of desArg⁹-BK is very low in the patient in a pathological situation (Sample 1) compared with the control.

FIG. 4B shows that when not in a pathological situation (Sample 3) the patient has a capacity to produce desArg⁹-BK comparable with that of the control.

The areas under curve AUCs in FIG. 4B represent the quantity of desArg⁹-BK released by activation of the contact phase; they are calculated using the trapeze method without smoothing.

| | Without smoothing |
|---|---|
| AUC 3B3 (patient in situation at rest, | 1299.2 ng/ml |
| Sample 3) | |
| AUC 3B1 (patient, pathological situation, | 125.6 ng/ml |
| Sample 1 | |
| Control | 957.9 ng/ml |

The calculation of the differences between the situation at rest (Sample 3) and the pathological situation (Sample 1) leads to knowledge of the production of desArg⁹-BK produced by activation of the plasma sample. This makes it possible to calculate the quantity of desArg⁹-BK (molecular weight 904) produced in the patient's sample volume during pathological development.

| | Without smoothing | |
|---|---|---|
| AUC 3B3 − AUC 3B1 | 1173.6 | ng/ml |
| (patient) | 1298 | nmol/l |

When related to the total plasma volume, the difference AUC 3B3—AUC 3B1 therefore corresponds to the quantity of desArg⁹-BK produced during the active phase of symptomatology:

| | Without smoothing | |
|---|---|---|
| AUC 3B3 − AUC 3B1 | 2.64 | mg |
| (patient) | 2920 | nmol |

The quantity of desArg⁹-BK produced is twice higher than the quantity of BK, which corresponds to the accumulation of desArg⁹-BK affected by an extended half-life.

1. Method for determining the quantity of BK antagonist needed to stop the effects of angioedema, using a plasma sample of the patient, comprising the following steps:
a) the release of BK is activated by contacting the patient's plasma sample with a determined quantity of electronegative agent;
b) an assay is performed of the quantity of BK released at step a), which corresponds to the BK reserve in the patient's plasma sample;
c) the value obtained at step b) is compared with a control value which corresponds to the BK reserve available in a plasma sample of a healthy control of same category, to determine the quantity of BK produced in the patient's plasma sample;
d) the total quantity of BK produced in the patient is determined; and
e) from the value obtained at step d) the necessary quantity of said antagonist is determined, this quantity being equimolar with the quantity of BK produced.

2. The method according to claim 1 wherein the assay at step b) is a competitive immunoenzymatic assay.

3. The method according to claim 1 wherein, for the assay at step b), use is made of anti-BK antibodies, DIG tracers coupled to BK, anti-DIG antibodies coupled to an enzyme and substrates of the enzyme.

4. The method according to claim 1 wherein step b) itself comprises the following steps:
anti-BK antibodies diluted in binding buffer are immobilised on a suitable support comprising several cavities;
at regular time intervals an aliquot portion of the activated sample is taken and said aliquot portion of the sample is incubated within a cavity of the support and a determined quantity of a DIG tracer coupled to BK, for a determined period of time;
after washing, anti-DIG antibodies coupled to an enzyme are incubated within the support;
after washing, a determined quantity of substrate of the enzyme is incubated within the support;
enzymatic activity is measured by direct colorimetric measurement.

5. The method according to claim 4 wherein the enzyme coupled to the anti-DIG antibodies is alkaline phosphatase or peroxidase.

6. The method according to claim 1 wherein the BK antagonist is icatibant (HOE 140).

7. The method according to claim 1 wherein the electronegative agent is chosen among dextran sulphate, glass beads and aluminium silicate.

8. A kit for implementing the determination method according to claim 1, comprising:
a first reagent consisting of anti-BK antibodies;
a second reagent consisting of a DIG tracer coupled to BK;
a third reagent consisting of anti-DIG antibodies coupled to an enzyme;
a fourth reagent consisting of a substrate of the enzyme.

9. A method for diagnosing gain-of-function angioedema in a patient, comprising:
a) activating the release of BK by contacting a plasma sample of the patient with a determined quantity of electronegative agent;
b) assaying of the quantity of BK released at step a), which corresponds to the BK reserve in the patient's plasma sample; and
c) comparing the value obtained at step b) with a control value which corresponds to the BK reserve available in a plasma sample of a healthy control of same category, to determine the amount of BK produced in the patient's plasma sample;
d) optionally, determining the total quantity of BK produced in the patient.
